(19) United States
(12) Patent Application Publication
(10) Pub. No.: US 2009/0196835 A1
(43) Pub. Date: Aug. 6, 2009

(54) TEETH WHITENING

(75) Inventor: Wyman Chan, London (GB)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(73) Assignee: SMILE STUDIO (UK) LTD, London (GB)

(21) Appl. No.: 12/301,110

(22) PCT Filed: May 16, 2007
(86) PCT No.: PCT/GB2007/001816

§ 371 Date: Mar. 27, 2009

(30) Foreign Application Priority Data
May 17, 2006 (GB) 0609784.4

(51) Int. Cl. A61K 8/22; A61K 8/41; A61Q 11/00

(52) U.S. Cl. 424/53; 424/54

(57) ABSTRACT

A composition for teeth whitening comprising: a bleaching agent; a acrylate; an amine wherein the amine may be selected from a group comprising R¹(NH₂)—CH₂OH; or H₂N—R²OH; or H₂N—R³OH—R⁴, where: R¹ represents CH₃—(CH₂)ₙ—CH, where n=0, 1, 2, 3, 4 or 5; R² represents (CH₂)ₙ, where n=2, 3, 4, 5, 6 or 7; R³ represents (CH₂)ₙ—CH, where n=0, 1, 2, 3, 4 or 5; and R⁴ represents CH₃ And the acrylate and amine may be initially present in two parts which, when mixed, provide a gel.

This invention relates to the whitening of teeth and specifically to compositions for use in the whitening of teeth. Whitening of teeth may be performed as an isolated procedure or, for example, following traditional teeth-cleaning methods or after dental procedures such as plaque removal.

The teeth of humans, as a species, display a wide range of shades of white. There is no one specific colour that a person's teeth are supposed to be.

However, it is known that teeth can become discoloured or darkened over time due to a variety of factors some of which relate to the ingestion of chromogenic factors such as tea, coffee, colas, red wine or smoking and some which relate to the ingestion of certain medicines (for example tetracycline) or excessive fluoride in young people. Some dental treatments (for example root canal work) can also lead to the darkening of individual teeth over time.

In order to combat the effects of darkening, bleach solutions (typically hydrogen or carbamide peroxide-based gels) have been proposed and used to remove stains and whiten teeth.

Conventionally, for home-whitening of teeth, a dentist takes a cast of a person's teeth and a tray of silicone rubber is fabricated from the cast. The dentist will instruct the person where they are to apply the bleach solution or gel to the cast. When the person is to whiten their teeth, the gel is placed in the tray at the appropriate locations and the tray is then inserted into the mouth and over the teeth. The excess gel will escape from the tray and onto the gum where it is wiped off to reduce or prevent irritation of the gum. The tray is left in place for a time to allow the gel to whiten the teeth.

In-practice, or chair-side, whitening usually follows the same steps, although a dentist may apply the gel to the teeth without using a tray, in which case a retractor may be used in the mouth to prevent the mouth from closing or to prevent the person from wiping the gel from the teeth. Laser or other intense light source bleaching can also be carried out in which a laser (or other light source) excites the bleaching compound to readily form oxygen radicals.

It is an object of the present invention to provide a composition for teeth whitening which promotes bleaching and/or stain removal and is at least as effective as known compositions. Further it may be more effective and/or may require a smaller financial outlay.

It is a preferred and/or non-exclusive object of the present invention to provide a method of whitening of teeth which is less likely to cause discomfort and/or pain to the individual having their teeth whitened.

It is a further preferred and/or non-exclusive object of the present invention to provide a method of whitening of teeth which may be carried out with or without using heat lamps or lasers and which is suitable for both chair-side and home use.

It is a yet further preferred object of the present invention to provide a bleaching composition which does not require transition or other metals to be used as activators and/or accelerators, although these may be present.

According to a first aspect of the invention there is provided a composition for teeth whitening comprising:
a. a bleaching agent;
b. an acrylate;
c. an amine

The composition is preferably a gel containing peptide bonds, e.g. peptide bonds formed between the acrylate and the amine.

A further aspect of the invention provides a composition for use in teeth whitening comprising a bleaching agent and an acrylate.

A yet further aspect of the invention provides a composition for use in teeth whitening comprising an amine.

The composition may comprise a bioactive glass.

The composition for teeth whitening may be formable by mixing:
a. a first solution including the bleaching agent and the acrylate; and
b. a second solution including the amine

The solutions may be aqueous solutions.

The bleaching agent may be hydrogen peroxide. The hydrogen peroxide may have very high purity, e.g. 98.5%. Preferably the concentration at which hydrogen peroxide is present in the first solution is 3-50 (v/v) %; for example 20-40% for chair-side use or 3-10% for home use. For example, a suitable solution may comprise 40 (v/v) % hydrogen peroxide, 48-49% of water and 1-2% of acrylate.

The acrylate may be a carbomer, such as Carbopol®.

The amine is preferably a primary or secondary amine, most preferably a primary amine. The amine may be an amino alcohol. The amino alcohol may have the general form R¹(NH₂)—CH₂OH; or H₂N—R²OH; or H₂N—R³OH—R⁴, where:

R¹ represents CH₃—(CH₂)ₙ—CH, where n=0, 1, 2, 3, 4 or 5;
R² represents (CH₂)ₙ, where n=2, 3, 4, 5, 6 or 7;
R³ represents (CH₂)ₙ—CH, where n=0, 1, 2, 3, 4 or 5; and
R⁴ represents CH₃

In addition to its role in gel formation, the amino alcohol preferably acts as an activator and/or accelerant for the bleaching agent. Preferably the concentration at which the amino alcohol is present in the second solution is 50-100%; more preferably 50-90%; most preferably 50-80%.

The second solution may also contain a particulate bioactive glass.

Any particulate bioactive glass is suitable. An example of a suitable material is NovaMin® supplied by NovaMin Technology Inc. (FL, USA). The purpose of the bioactive glass is to reduce teeth sensitivity and therefore the possibility of the user experiencing pain or discomfort during teeth whitening. NovaMin achieves this by forming hydroxyapatite on a surface of a tooth, which blocks dentin tubules and reduces tooth sensitivity.

According to a fourth aspect of the invention there is provided a method of whitening, e.g. cosmetic whitening, of teeth comprising mixing a bleaching agent, an acrylate and an amine to form a composition for teeth whitening and applying the so-formed composition to at least one tooth.

The composition for teeth whitening may be applied to at least one tooth following any of the methods set out in our co-pending British patent application, published as GB 2416309 (the entire contents of which being incorporated by reference herein).

A two-barreled syringe may be used to apply the composition for teeth whitening to at least one tooth, the syringe containing in a first barrel a pre-mixed solution containing a bleaching agent and in a second barrel a pre-mixed solution containing an amine. The two solutions may be applied in a range of ratios from 1:5 to 5:1; preferably between 1:4 and 4:1.

Typically each application of the composition for teeth whitening lasts for 5 to 20 minutes, say 5 to 10 minutes. A teeth whitening regime may consist of one or more applications; preferably 2-5 applications. Nevertheless it is emphasised that the application regime for each person may be tailored to their specific requirements, depending on, among other things, the severity and extent of staining and/or discolouration present, the person's desired final teeth whiteness, the sensitivity of the person's teeth, the amount of time and money the person can afford.

The composition for teeth whitening may be applied at ambient temperatures and is effective at such temperatures. Conveniently no irradiation, e.g. from a heat source, a lamp or a laser is required, although the use of such means is not discounted. The fact that heat sources, lamps or lasers are not necessary may reduce financial outlay for the user.

Advantageously no specific preparatory work is generally required to prepare a tooth for the application of the composition for teeth whitening. Furthermore, no post-application procedure need be adhered to. However, as is usual practice, it may be preferable to ensure that the teeth surfaces are clean and preferably free of plaque before commencing whitening.

In order that the invention may be more fully understood, it will now be described by way of example only, and with reference to the accompanying drawings, in which:

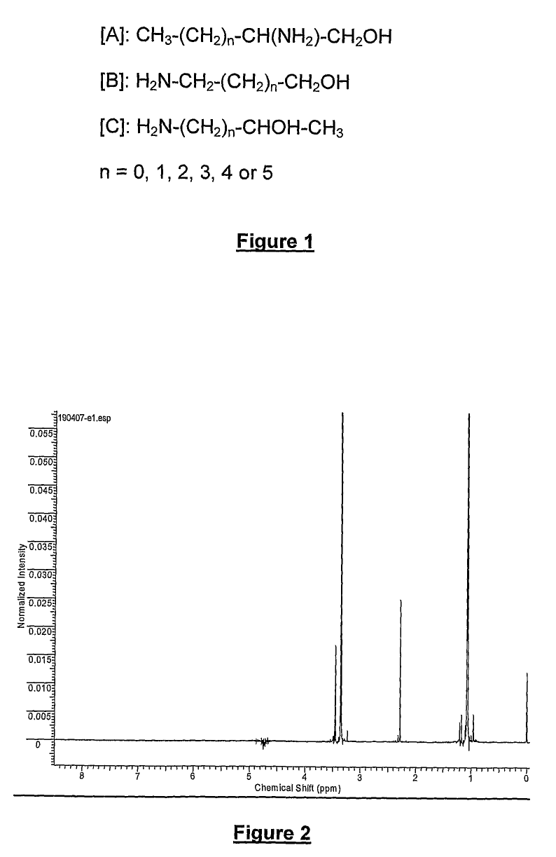

FIG. 1 shows molecular structures of suitable amino alcohols;

FIG. 2 shows an NMR-¹H spectrum of an amino alcohol;

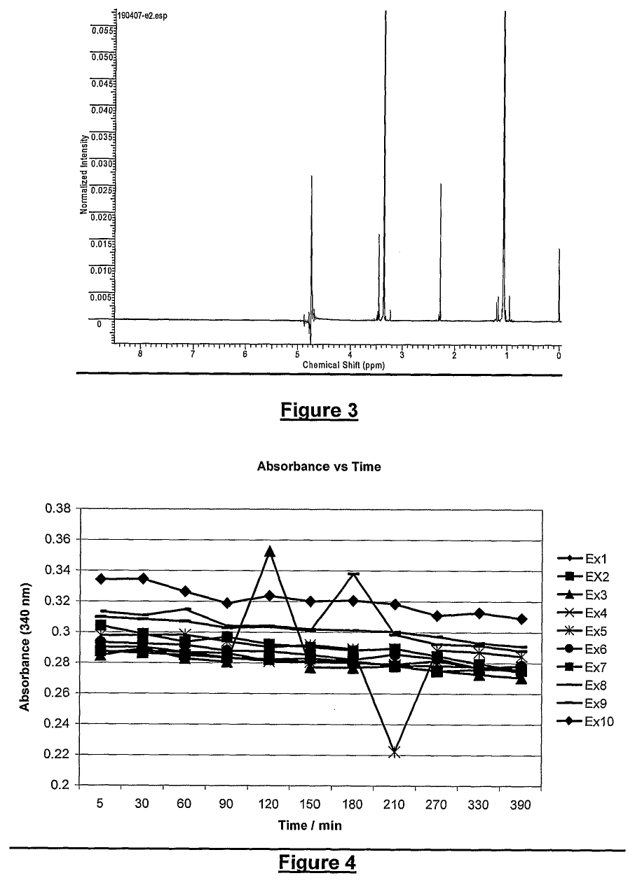

FIG. 3 shows an NMR-¹H spectrum of the amino alcohol of FIG. 2 treated at 60° C.;

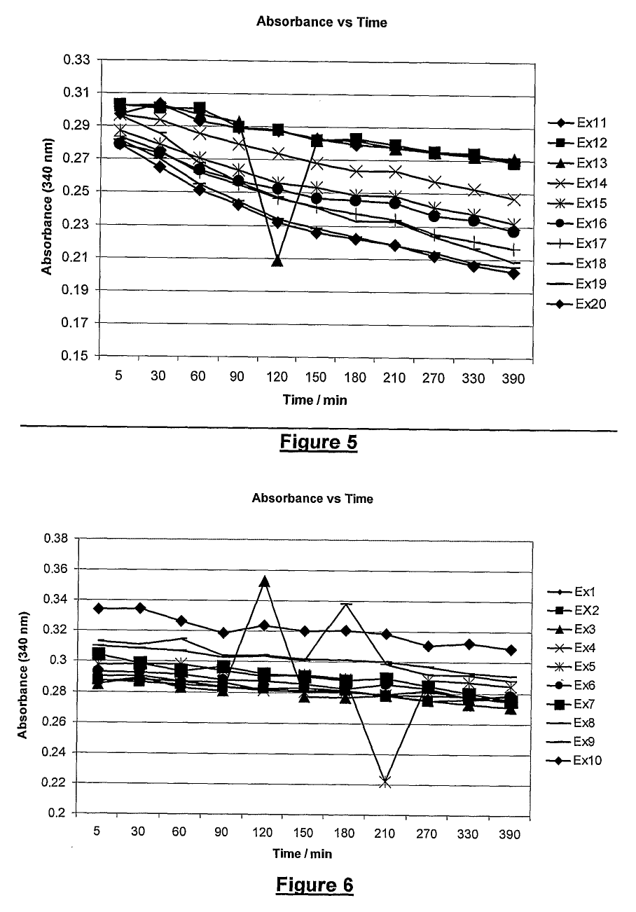

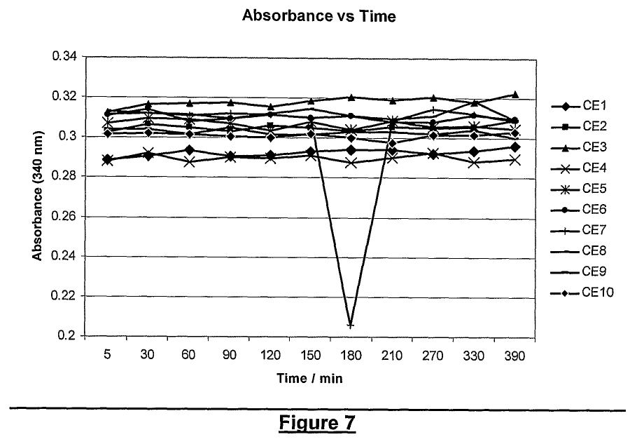

FIGS. 4 to 6 provide graphical data corresponding to Examples 1 to 30; and

FIG. 7 provides graphical data corresponding to Comparative Examples 1 to 10.

A pre-mixed aqueous solution containing 40 (v/v) % hydrogen peroxide (98.5% pure) and 1.5% carbopol is put into a first barrel of a two-barrelled syringe. A second pre-mixed aqueous solution containing an amino alcohol (80%), (B, n=2) selected from those depicted in FIG. 1, and a spectrum of which is shown in FIG. 2, and NovaMin (20%) is put into a second barrel of a two-barrelled syringe. The two aqueous solutions are mixed in a 4:1 ratio to provide a gel composition for teeth whitening.

The gel is formed when the first and second solutions are mixed together. In particular, the gel is formed as a result of a reaction between the acrylate in the first solution and the amine in the second solution. The reaction between the acrylate and the amine forms a polypeptide gel.

The so-formed gel is applied to at least one tooth for a period of, say, 10 minutes. This procedure is repeated on four separate occasions.

The viscosity of the gel is sufficient to ensure that once applied, the gel remains on tooth surfaces.

The increased bleaching and/or stain removal effectiveness of the inventive composition compared with existing products has been demonstrated in tests on tea-stained paper.

Without wishing to be constrained by theory the potentially superior performance of the inventive composition is thought to be due to the high purity of the hydrogen peroxide contained in the composition; and furthermore the use of an organic activator, e.g. an amino alcohol, instead of a transition metal is thought to promote formation of more effective bleaching species.

FIG. 3, which was recorded subsequent to treatment at 60° C. to the amino alcohol shown in FIG. 2, indicates that there are structural changes to the amino alcohol subsequent to contact with hydrogen peroxide

A series of experiments have been completed in which a stain solution is treated with various peroxide and amino alcohol solutions to demonstrate efficacy of the combination of amino alcohol and peroxide.

To do so, melanoidins were prepared in aqueous solution (5.00×10⁻² mol·dm⁻³ phosphate buffer, pH 7.0) by incubation of equimolar mixture of L-lysine and D-glucose (5.00×10⁻³ mol·dm⁻³ each) for 24 hours at 60° C. 1.0456 ml aliquots of the obtained brown melanoidin solution were then used to prepare thirty 10 ml samples of an equal initial absorbance value.

The samples were arranged into three groups accordingly to the hydrogen peroxide concentration (10 mM, 50 mM and 100 mM), with each group including an activator-free blank as an internal control and 9 samples containing increasing levels of amino alcohol activator, as follows:

TABLE 1
Concentration Data for Stain Removal Experiments

| Example | [A]/mM | [Peroxide]/mM | [A]/[Peroxide] |
|---|---|---|---|
| 1 | 0 | 10 | — |
| 2 | 0.5 | 10 | 0.05 |
| 3 | 1 | 10 | 0.1 |
| 4 | 2 | 10 | 0.2 |
| 5 | 4 | 10 | 0.4 |
| 6 | 5 | 10 | 0.5 |
| 7 | 8 | 10 | 0.8 |
| 8 | 10 | 10 | 1 |
| 9 | 15 | 10 | 1.5 |
| 10 | 20 | 10 | 2 |

TABLE 2
Concentration Data for Stain Removal Experiments

| Example | [A]/mM | [Peroxide]/mM | [A]/[Peroxide] |
|---|---|---|---|
| 11 | 0 | 50 | — |
| 12 | 2.5 | 50 | 0.05 |
| 13 | 5 | 50 | 0.1 |
| 14 | 10 | 50 | 0.2 |
| 15 | 20 | 50 | 0.4 |
| 16 | 25 | 50 | 0.5 |
| 17 | 40 | 50 | 0.8 |
| 18 | 50 | 50 | 1 |
| 19 | 75 | 50 | 1.5 |
| 20 | 100 | 50 | 2 |

TABLE 3
Concentration Data for Stain Removal Experiments

| Example | [A]/mM | [Peroxide]/mM | [A]/[Peroxide] |
|---|---|---|---|
| 21 | 0 | 100 | — |
| 22 | 5 | 100 | 0.05 |
| 23 | 10 | 100 | 0.1 |
| 24 | 20 | 100 | 0.2 |
| 25 | 40 | 100 | 0.4 |
| 26 | 50 | 100 | 0.5 |
| 27 | 80 | 100 | 0.8 |
| 28 | 100 | 100 | 1 |
| 29 | 150 | 100 | 1.5 |
| 30 | 200 | 100 | 2 |

The data according to each of the thirty experiments is shown in FIGS. 4 to 6.

Following the addition of hydrogen peroxide, the absorbance of the bleached samples and the controls was monitored at 340 nm for 390 minutes using Cobas Mira Chemistry System selective analyser. The rate of the bleaching process, defined as the decrease in the absorbance achieved at the end of each treatment period, was determined for each sample. Multifactorial analysis of variance of the generated data shown that accelerating properties of amino alcohol activator added to hydrogen peroxide solution had highly significant effect on the rate and extent of the bleaching process, demonstrating the ability of the activator to enhance bleaching properties of hydrogen peroxide.

In order to demonstrate that the amine had exhibited no bleaching activity in the absence of peroxide a series of comparative examples were run in which stain solutions were exposed to various concentrations of amino alcohol. The data is shown in Table 4 below and in FIG. 7.

TABLE 4
Comparative Data

| Comparative | | |
|---|---|---|
| Example | [A]/mM | [Peroxide]/mM |
| 1 | 0 | 0 |
| 2 | 5 | 0 |
| 3 | 10 | 0 |
| 4 | 20 | 0 |
| 5 | 40 | 0 |
| 6 | 50 | 0 |
| 7 | 80 | 0 |
| 8 | 100 | 0 |
| 9 | 150 | 0 |
| 10 | 200 | 0 |

Clearly, the amino alcohol, in isolation, had no effect on the stain solution.

In order to compare the efficacy of compositions of the invention compared to leading commercially available products a further series of experiments were conducted.

Model brown-coloured melanoidins were prepared in aqueous solution (5.00×10⁻² mol·dm⁻³ phosphate buffer, pH 7.0) by incubation of an equimolar mixture of L-lysine and D-glucose (5.00×10⁻³ mol·dm⁻³ each) for 20 hours at 60° C.

For comparative evaluation, 1 g of each bleaching product and a hydrogen peroxide-free control Carbopol® gel (2% w/w) were subjected to dissolution in 3.5 ml aliquots of the obtained brown solution re-heated to 35° C. A control comprising no hydrogen peroxide was also prepared. The break-down and dissolution of the gels in aqueous solution was achieved by centrifugation of the mix for 5 minutes at 5000 rpm.

Following centrifugation, the samples were incubated at 35° C. for 1 hour. 3 ml aliquots of each incubation mixture were then centrifuged at 5000 rpm for further 15 minutes to clarify and degas the solution.

Subsequent to diluting each sample 1:9, the absorbance of the bleached solutions and the control was measured at 325 nm. The bleaching efficacy, defined as decrease in the absorbance achieved after 1 hour treatment at orally-relevant temperature, was determined for each product with respect to the inactive control sample. The decrease in the absorbance per hour per 1% (w/w) of peroxide was calculated for each product as shown below in Table 5.

TABLE 5
Commercially Available Solutions and
a Composition of the Invention

| Product | CE11^{a} | CE12^{b} | Example 31 | Control |
|---|---|---|---|---|
| Initial H₂O₂ | 20% | 25% | 20% | 0% |
| concentration | | | | |
| (w/w) | | | | |
| H₂O₂ | 5.71% | 7.14% | 5.71% | 0% |
| concentration in | | | | |
| dissolved sample | | | | |
| (w/w) | | | | |
| Sample | 0.733 | 0.674 | 0.486 | 1.160 |
| Absorbance at | | | | |
| 325 nm after 1 hr | | | | |
| treatment | | | | |
| ΔA₃₂₅ (units/hr) | 0.427 | 0.486 | 0.674 | 0 |
| ΔA₃₂₅ (units/[hr × | 0.0748 | 0.0681 | 0.1180 | N/A |
| 1% H₂O₂]) | | | | |

^{a}Zoom! Obtainable from Discus Dental Inc. of Shenley Hertfordshire, UK
^{b}Zoom 2 obtainable from Discus Dental Inc. of Shenley Hertfordshire, UK

It has therefore been demonstrated that an amino alcohol in concert with hydrogen peroxide provides a more effective bleaching solution. The use of such compounds on teeth has been carried out and showed improved bleaching performance over hydrogen or carbamide peroxide in isolation, or in the presence of transition metal activators.

Moreover, the combination of acrylates with the amino alcohol is a ready means of preparing a gel solution for the application to teeth. The use of a double-barrelled syringe facilitates the application, although is not necessary.

The invention is not limited to the use of hydrogen peroxide, carbamide peroxide may be used in its stead or in addition thereto. Also, other species which form hydrogen peroxide in situ may be used, bearing in mind the toxicology of the species and of any side reactions.

1-16. (canceled)

17. An aqueous teeth whitening composition comprising:
a. a bleaching agent;
b. an acrylate; and
c. an amino alcohol
wherein the amino alcohol is a primary or secondary amino alcohol, has a carbon chain of from three to eight carbon atoms and is provided in molar excess of the acrylate.

18. A composition according to claim 17, wherein the composition is in the form of a gel formed between the acrylate and amine.

19. A composition according to claim 17, wherein the volume ratio of amino alcohol to acrylate is between 5:1 and 500:1.

20. A composition according to claim 17, wherein the amino alcohol comprises a primary amine having four carbon centers.

21. A composition according to claim 17, wherein the amino alcohol has the general formula R¹(NH₂)—CH₂OH; or H₂N—R²OH; or H₂N—R³OH—R⁴, where:
R¹ represents CH₃—(CH₂)ₙ—CH, where n=1, 2, 3, 4 or 5;
R² represents (CH₂)ₙ, where n=3, 4, 5, 6 or 7;
R³ represents (CH₂)ₙ—CH, where n=1, 2, 3, 4 or 5; and
R⁴ represents CH₃.

22. A composition according to claim 17, wherein the bleaching agent is hydrogen peroxide.

23. A composition according to claim 17 further comprising a bioactive glass.

24. A method of whitening teeth comprising applying a composition according to claim 17 to at least one tooth.

25. A teeth whitening composition comprising:
a. a bleaching agent;
b. an acrylate; and
c. an amine,
wherein the amine is a primary or secondary amine and wherein the amine is provided in molar excess of the acrylate.

26. A composition according to claim 25, wherein the amine is an amino alcohol.

27. A composition according to claim 25, wherein the amine has a carbon chain of from three to eight carbon atoms.

28. A composition according to claim 25, wherein the composition is in the form of a gel formed between the acrylate and amine.

29. A composition according to claim 25, wherein the volume ratio of amine to acrylate is between 5:1 and 500:1.

30. A composition according to claim 25, wherein the bleaching agent is hydrogen peroxide.

31. A composition according to claim 25, further comprising a bioactive glass.

32. A method of whitening teeth comprising applying a composition according to claim 25 to at least one tooth.

33. A two-part teeth whitening composition comprising a first solution including a bleaching agent and an acrylate and a second solution including an amine, wherein in use the first and second solutions are mixed together in a ratio between 1:5 and 5:1 to form a polypeptide gel, wherein the amine is a primary or secondary amine, and wherein the amine is provided in molar excess of the acrylate.

34. A composition according to claim 33, wherein the amine is an amino alcohol.

35. A composition according to claim 33, wherein the amine has a carbon chain of from three to eight carbon atoms.

36. A composition according to claim 33, wherein the ratio of amine to acrylate is between 5:1 and 500:1.

37. A composition according to claim 33, wherein the bleaching agent is hydrogen peroxide.

38. A composition according to claim 34, wherein the amino alcohol comprises a primary amine having four carbon centers.

39. A composition according to claim 34, wherein the amino alcohol has the general formula R¹(NH₂)—CH₂OH; or H₂N—R²OH; or H₂N—R³OH—R⁴, where:
R¹ represents CH₃—(CH₂)ₙ—CH, where n=1, 2, 3, 4 or 5;
R² represents (CH₂)ₙ, where n=3, 4, 5, 6 or 7;
R³ represents (CH₂)ₙ—CH, where n=1, 2, 3, 4 or 5; and
R⁴ represents CH₃.

40. A composition according to claim 33, wherein the first solution and second solution are mixed in a ratio between 1:4 and 4:1.

41. A composition according to claim 33, wherein the bleaching agent is present at a concentration of 3-50 volume/volume % of the first solution.

42. A composition according to claim 33, wherein the amine is present at a concentration of 50-100 volume/volume % of the second solution.

43. A composition according to claim 33, wherein the acrylate is present at a concentration of 1-2 volume/volume % of the first solution.

44. A composition according to claim 33, further comprising a bioactive glass.

45. A method of whitening teeth comprising applying a composition according to claim 33 to at least one tooth.

46. A method of whitening teeth comprising the steps of:
mixing a first solution including a bleaching agent and an acrylate with a second solution including an amine in a ratio between 1:5 and 5:1 to form a polypeptide gel wherein the amine is a primary or secondary amine, and wherein the amine is provided in molar excess of the acrylate; and
applying the polypeptide gel to at least one tooth.

47. A method according to claim 46, wherein the amine is an amino alcohol.

48. A method according to claim 46, wherein the amine has a carbon chain of from three to eight carbon atoms.

49. A method according to claim 46, wherein the ratio of amine to acrylate is between 5:1 and 500:1.

50. A method according to claim 46 further comprising use of a dual-barrelled syringe, wherein the first solution is located in a first barrel of the syringe and the second solution is located in a second barrel of the syringe.
